(19) United States
(12) Patent Application Publication
(10) Pub. No.: US 2010/0193709 A1
(43) Pub. Date: Aug. 5, 2010

(54) Apparatus and Method for Sanitizing Feet and the External Surfaces of Footwear

(76) Inventor: Linda M. Dalton, Singapore (SG)

Correspondence Address: DONN K. HARMS;PATENT & TRADEMARK LAW CENTER, SUITE 100, 12702 VIA CORTINA, DEL MAR, CA, 92014, US

(21) Appl. No.: 12/365,630

(22) Filed: Feb. 4, 2009

(51) Int. Cl. G01J 3/10

(52) U.S. Cl. 250/504 R

(57) ABSTRACT

A disinfecting device for objects such as shoes or human body parts such as feet. The device employs light radiating from a cavity below a transparent platform, to contact and disinfect surfaces on objects placed on a top surface of the platform. The spectral range of the light is adapted to disinfect the surface with Ultra Violet light being a preferred spectral range. An upper housing may be provided above the platform to protect the user's eyes from UV radiation which is substantially indiscernible to the human eye and as a housing for a reflective means to reflect disinfecting light communicating through the transparent platform, back toward the top surfaces of the object on the platform. A signal component for user safety such as a visible light emitter, an audible alarm, or vibration of the platform may be employed to provide the user a means to ascertain emanating UV light during operation of the device.

### BACKGROUND OF THE INVENTION

1. Field of the Invention

The present invention relates generally to sanitizing devices. More particularly, the device and method herein disclosed and described relate to an apparatus and a method used for easily, quickly and effectively sanitizing the outer bottom surfaces of footwear or feet and of other items, such as hand bags, purses, backpacks and the like, which may have been exposed to micro organisms such as bacteria, fungi, yeasts, and viruses and the like.

Using the preferred embodiment of the present invention, which employs germicidal radiation technology to accomplish the disinfection, the outer soles and heels of footwear may be quickly and effectively disinfected while still on the feet of the wearer. In use, germicidal radiation, such as ultraviolet light, bathes the feet or footwear or other item to be disinfected for a defined period of time, when the device is weight-activated or activated through other means.

2. Description of Related Art

The present invention is concerned with the problem of pathogenic microorganisms carried on the outer bottom surfaces of footwear or of other items that have been exposed to unsanitary areas or the problem of pathogenic microorganisms that infect the surface of the feet.

The outer bottom surfaces of footwear, principally the outer soles and heels, can come into contact with floor areas or outdoor ground areas that may be unsanitary and contaminated with microorganisms such as bacteria, viruses, molds, fungi and yeasts. Areas where such microbial contamination commonly exists include public toilets, public sidewalks and streets, parks, farms, etc. resulting from urine in areas near public toilets and urinals, animal urine and feces as well as human sputum on sidewalks, streets, lawns, etc.

The outer bottom surfaces of footwear which come into contact with such contaminated areas also become contaminated with microorganisms and thereby become a source of further microbial contamination. As a consequence, footwear entering residential premises with such contaminated footwear carries the pathogenic microorganisms into the residence onto floors, carpeting, closets, shoe racks, etc.

Further, when handling the footwear, for the purpose, for example, of removing the footwear, polishing the footwear or, placing the footwear into shoe racks, touching the outer bottom surfaces of the footwear, i.e. the soles or heels, will result in the transfer of the pathogenic microorganisms from the footwear, onto the hands of the person handling it. Subsequently, they can easily communicate it into the body through subsequent touching of the mouth, eyes, ears, and such, thereby causing infection. Similarly, putting feet up on tables will cause contact contamination and a further spread of pathogenic germs. As such, toddlers crawling on the floor or carpets can be easily infected through hand, face, and body contact with the contaminated floor and carpets.

Similar contamination problems exist at commercial venues. Especially commercial livestock and agriculture enterprises which are subject to diseases that can be transmitted to the livestock or plants by visitors are vulnerable and require protection against such contamination that can be introduced by footwear.

Still further, as a consequence of modern air travel security regulations, the potential for user contact with germs and other pathogens has been increased because carry-on luggage must be continually kept in the traveler's presence. As a consequence, travelers modernly must take their carry-on luggage into airport bathrooms and leave them upon the floor where a plethora of domestic and international pathogens may reside. Thereafter, carrying the luggage onto the plane requires contact with their hands, especially while placing and removing it from the overhead storage.

In order to solve this problem, some prior art teaches the provision of disinfection devices which are intended to be placed at the entrance of home and business premises. However, all of these devices have as a drawback that they require the use of cleansing liquids, liquid disinfectants or spray-on disinfectants, which wet the bottom of the footwear. These liquids can be slippery and lead to falls or can be tracked into the premises causing stains or dirt spots on the flooring or tracking potentially toxic disinfectants into the residence.

Some of these conventional devices also require the use of scrapers or brushes, either manual or mechanical, as well as troughs, with or without a spongy material, to contain and apply the disinfectant. These devices have the disadvantage that they are rather cumbersome, unwieldy and require maintenance such as the refilling of the liquid cleansers or disinfectants, replacement of the brushes, replacement of the spongy material, etc.

Pathogenic microorganisms can also come into contact with bare feet when they are exposed to such organisms. The most common type of infection of the feet is caused by pathogenic fungi that invade the dead tissue cells at the outer skin surface of the feet. The best known of such superficial fungal infections is athlete's foot, also called Tinea pedis, and therefore discussion of superficial fungal infections of the feet in this invention will be confined to athlete's foot for reasons of simplicity although the present invention and its utility also relate to other forms of infections of the feet. Athlete's foot infection causes itching, burning and often blisters. In addition to discomfort, it provides sites for more serious infections. It is a contagious infection. Its spread has been traditionally associated with the floors of any place where people customarily go barefoot whether it be the family bathroom or locker room at the local golf club, public swimming pool or school gymnasium, or in hotel rooms.

Various methods have conventionally been employed to treat athlete's foot. Currently the most common treatment method is chemical therapy, typically consisting of the application of an antifungal agent, possibly in combination with other chemicals. Other methods include application of heat to the affected part by hot water, infrared lamp or laser irradiation or other devices using combinations of chemicals and physical approaches.

Such chemical treatment therapies, can be applied in the form of many types of powders, sprays, solutions, lotions or creams which have been made available for treatment. These often lack effectiveness due to poor delivery of the medication to the source of the condition, to loss of the medication through abrasion from normal activity of the patient, to absorption of medications applied to the skin by the patient's socks or clothes. The medications for these conditions typically require at least two to four weeks of continuous treatment, and thus often fail due to this poor delivery.

A further problem with the use of existent medications is the lack of compliance by patients who employ them. Due to the mess and difficulty of use, patients will often use such over the counter medications only until their symptoms abate. They then will stop using the medication, before the recommended course of treatment is completed and hence often before the infection has truly cleared. Though momentarily abated, the infection then begins to take hold again, and in a matter of days or weeks a full-blown infection occurs again. Since the surviving organisms are those that are resistant to the medication, the subsequent infection can become harder to treat. In many cases the patient will repeat using the over-the-counter medication until the symptoms clear, and again stop the medication with the first sign of abatement, with the whole cycle repeating and leading to chronic athlete's foot.

Other treatment methods using heat or irradiation often require complex, cumbersome or expensive equipment and procedures which may also be complex and not easily applied. Further, doctors can be hesitant to prescribe such treatments for fear of patient mistakes as to their application and possible injury or other consequences.

### REFERENCES SITED

United States Patent No. 20080104782 (Hughes) discloses a fluid-based cleaning and disinfecting system for the cleaning and disinfecting of footwear or feet. An optional brush on a wand provides cleaning to upper foot areas as required. Fluid is dispensed when a user is positioned on the device, thereby minimizing the volume of fluid required to perform the process. Optionally, a venturi component allows the concurrent employment of a cleaning or disinfecting fluid with the cleaning solution or water. When the system is attached to a hot water line and fitted with heating elements, it can be used to remove snow from shoes and boots. However the Hughes device requires liquid cleansers and drainage systems and is most inconvenient to users not wishing to wet their footwear or feet.

U.S. Pat. No. 4,425,677 of (Cox) teaches a shoe cleaner for removing debris from the sole of a shoe that is comprised of a foraminous scraper for contacting the sole of a shoe and, upon relative motion therebetween, removing debris therefrom. Also disclosed is a wick member for applying a liquid deodorant and/or solvent for the debris to the shoe from a reservoir proximate to the foraminous scraper. The shoe cleaner disclosed herein also preferably includes a mat for removal of any excess deodorant and/or solvent and for secondary removal of debris from the sole of the shoe. The Cox device requires the user to scrape and use liquids in the process which can be inconvenient as noted. The scraping action may also require the user to balance on one foot while cleaning the other which is not conducive for use by overly young or old users.

U.S. Pat. No. 4,922,578 (Miettinen) describes a shoe sole cleaner which employs a power section and a brush section. The brush section is essentially plane like and it includes brush rods. The power section and the brush section are placed at a distance from each other. The power section is provided with a fluid power supply. The brush section includes a vibrating member such as a piston in a piston and cylinder combination. The fluid power supply is connected to the vibrating member by a fluid transmission means such as a pipe. The vibrating member is in contact with the brush rods and by means of the vibrating member the brush rods are made to move in a desired back and forth motion. The shoe sole cleaner according to the invention is low in construction so that it fits in the place of ordinary doormats. The cleaner can be extended simply by connecting several similar brush sections together as a large shoe sole cleaner. Miettinen is far too complicated in structure and use for wide deployment in homes and businesses and requires a fluid supply and would tend to be noisy due to the vibration required.

U.S. Pat. No. 5,950,269 (Openshaw) teaches a sole cleaning device for cleaning the bottom and sides of the soles of boots and shoes. Openshaw includes a rotating brush mounted above a container of water so that upon rotation of the brush a portion of the distal ends of the brush extend into the water. The device further includes a removable scraper grate for supporting a shoe above the water but in engagement with the brush. A motor for rotating the brush against the sole of the boot. The removable grate is provided with a pair of spaced and parallel side rails having fixed brushes mounted to the side rails and facing one another. The removable grate further includes a sieve platform extending on one side of the grate above the water. Sponges are positioned on top of the sieve for removing water from a boot or shoe placed on the sponge. As can be discerned Openshaw is another fluid-based device and includes motors and rails all of which complicate its use.

U.S. Pat. No. 6,557,203 (Meshbesher) discloses an apparatus for cleaning and sanitizing footwear including a container for disinfectant liquids which has an upwardly open U-shaped channel tack-welded to the bottom panel of the container. At least one brush is removably attached to operate in the channel to remove debris from the bottom of footwear while a pair of inwardly facing brushes concurrently clean the sides of the footwear. Preferably, a scraper blade is attached to one end panel of the container. Outwardly extending flanges are provided on opposed surfaces of the container for manual grasping and lifting of the system. Optionally, a pair of laterally extending L-shaped brackets attached to opposite sides of the container are provided for stabilizing the system during use. Meshbesher as such, much like the other art, employs fluids and complicated mechanical elements which increase cost and limit use.

U.S. Pat. No. 6,584,636 (Schlem) describes a footwear cleaning apparatus that includes a vertically oriented frame with an upper frame for supporting the user that is standing while cleaning the footwear. The apparatus receives water from a garden hose and provides a control valve for regulating the water flow while the user is standing upright. The water is directed upwardly through a foot platform on the lower frame and through a lower brush onto the footwear sole, the resulting splash causing water to wet two side brushes that are oriented to bear upon the sides of the footwear. A scraper assembly is also on the lower frame and provides a horizontal scraping edge and two vertical scraping edges for the sole and sides of the footwear, respectively. After the water is turned off, a drain outlet allows the system to be drained. Schlem consequently employs fluids and required drainage and a scraper, all of which limit wide deployment and use.

U.S. Pat. No. 4,640,283 (Shigeki) describes a method for curing athlete's foot based on applying irradiation to the effected skin area using a laser beam of very specific type. Shigeki requires use under very concise conditions of laser energy density and very accurate timing in milliseconds of exposure to produce a thermal treatment. However the thermal treatment must be maintained within a very specific temperature range in order to kill the fungus causing the athlete's foot infection, and not injure the user in the process. Hazards of the Shigeki device thus hinder use and deployment widely.

U.S. Pat. No. 5,819,431 (Harold) discloses a foot dryer apparatus and method of drying feet based on dispensing heat under pressure over the entire upper and lower portions of the foot of the user. Concurrently with the heat, a mist of powder particles is dispensed to the foot with the aim of using heat and drying to inhibit the athlete's foot infection. Harold thus replaces fluids with sprayed powder which must be reloaded and can become airborne which if not a health hazard can become a detriment to use since the powder would settle in the room where it is used in other places.

As such, there is an unmet need for a simply employed device to kill potential pathogens which may occupy the surfaces of footwear and the user's feet themselves. Such a device and method should not require fluids which might limit use against skin and must be refilled and require more complicated components. Such a device should require little if any input or attention from the user to avoid injury thereby encouraging its use, and its recommendation for use by physicians. Such a device should also be adapted to disinfect small articles such as shoe bottoms, or luggage bottoms, which are subjected to some of the most dirty environments possible and hence carry the worst pathogens.

However, none of the conventional efforts provide the benefits attendant with the present disclosed device and method. The present invention achieves its intended purposes, objects and advantages over the prior art devices through a new, useful and unobvious combination of method steps and component elements with the use of a minimum number of functioning parts and by employing readily available materials thereby yielding a reasonable cost to manufacture.

In this respect, before explaining at least one embodiment of the invention in detail it is to be understood that the invention is not limited in its application to the details of construction and to the arrangement, of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for designing of other structures, methods and systems for carrying out the several purposes of the present invention. It is important, therefore, that the claims be regarded as including such equivalent construction insofar as they do not depart from the spirit and scope of the present invention.

### SUMMARY OF THE INVENTION

The disclosed device and method provide for disinfecting the outer external bottom surfaces of footwear and other items and/or disinfecting feet of the user by the employment of radiation sterilization. Different methods exist to sterilize using radiation, and these comprise electron beams, X-rays, gamma rays, subatomic particles and ultraviolet irradiation. Radiation sterilization is a relatively newer technology to provide disinfection/sterilization without the use of liquid or spray disinfectants. Ionizing radiation, such as gamma rays, is used for the disinfection of foods, medical hardware, etc. Irradiation is also used by the United States Postal Service to sterilize mail.

Ultraviolet light irradiation using germicidal ultraviolet UV-C lamps with wavelengths in the 100-280 nm range is particularly useful for sterilization of surfaces if properly projected thereon for the proper amount of time. UV irradiation is routinely used to sterilize the interiors of biological safety cabinets. In the preferred embodiments of the device and method herein, ultraviolet germicidal radiation is employed as a sterilization radiation source. It is preferred due to the fairly common availability of this technology, and the relatively low cost enabling the overall cost of the device to be kept low. Further, since many users are easily distracted from what they are doing at any given time, UV irradiation would be one of the safest of the various radiation options available. This has to do with the inability of the UV-C rays to penetrate the body's surfaces. UV-C has an extremely low penetrating ability and thus it is anticipated that it is substantially completely absorbed by the outer dead layer of the skin (stratum corneum) on the user's skin where it does little harm. Since this is the area of the skin in which superficial fungal infections are generally located, UV-C radiation is especially preferred as it can effectively kill the causative fungal organisms that colonize the dead skin cells at the surface of the feet.

In the case of contamination of the bottom surface areas of footwear and other items such as handbags and carry-on luggage which come into contact with very contaminated surfaces at airports and public places, bacteria, yeasts, fungi and viruses, will in time become resident on the surface of the exposed footwear and other items. Since disinfecting an exposed surface does not require penetrating radiation the employment of UV-C radiation effectively kills these microorganisms on the radiation-exposed surfaces, while concurrently allows for use of the disclosed device on skin.

An additional method of operation for the current invention is an improved method for the disinfection of the feet of livestock. Currently known art makes use of more complicated methods of walking the livestock through troughs containing liquid disinfectants or over absorbent pads impregnated with disinfectant solutions. This requires the need to wet the feet of the livestock and requires regular refilling and maintenance and the use of liquid pesticidal disinfectant chemicals. The disclosed device and method may be easily adapted in size to accommodate livestock and perform disinfection of the feet without complicated systems requiring disinfectant solutions, troughs, absorbent pads, and requisite maintenance.

As disclosed herein, the apparatus provides for the quick and easy disinfecting of the bottom outer surfaces of footwear, principally the outer soles and heels, that come into contact with floor and/or ground surface areas that may be unsanitary and contaminated on outer surfaces with microorganisms such as bacteria, viruses, molds, fungi and yeasts. Thus, the footwear outer surfaces contaminated with microorganisms and which have become a source of further microbial contamination, may be cleaned of this perilous health problem by the user without the annoying requirement of removal of the footwear from the feet.

In one preferred embodiment, the apparatus includes a housing which may resemble a bathroom scale and employs a substantially transparent top platform portion to stand upon. In use, one or a plurality of ultraviolet germicidal lamps operatively mounted inside the housing and engaged to a power source, are activated to kill the microorganisms on the user's feet or shoes. Activation to energize the lamps is in the preferred mode of the device herein, provided by a weight-activated switch that connects the ultraviolet lamps to the power source. Alternatively a manual switch that actuates the ultraviolet lamps may be employed. Additionally, and preferred, a timer switch that turns off the power to the ultraviolet light source, after the required exposure time, may be employed in combination with either or both of the weight-activated or manual means for switching.

The device may also employ safety features to protect users mounting it, against prolonged eye exposure and to protect users such as unsupervised children from harm. One such safety feature is the employment of transparent tempered glass or polymer panel or other suitable material for the transparent top platform. Forming the top platform from such material provides a means to block a large proportion of the UV-C radiation which might escape as stray light. Further, the material can reduce the intensity of the UV-C radiation transmitted through the clear platform using commercially available bulbs so that it is sufficient to be germicidal to microorganisms present on the external surfaces of footwear, other items, or feet placed on the clear plate platform after the required exposure length, but without harmful effects to the users of the apparatus.

In addition to the composition of the material used for the transparent platform, means for controlled transmission levels of the UV-C radiation through the clear platform can also be achieved by one or a combination of such means including adjustment of the thickness of the clear plate, the radiating power of the UV-C source, employing a specific wavelength in the 100 to 280 nm range, the use of UV-C absorbent film on the bottom side of the clear plate platform.

Although these safety features are novel in their application to the device and method herein those skilled in the art will no doubt realize other means to control radiation transmission upon reading this disclosure. As such, any such means to control radiation transmission as would occur to those skilled in the art, is anticipated within the scope of this application.

In use as a method of disinfecting a surface, the apparatus is placed on the floor in an area immediately prior to entering the area that is meant to be protected from microbial contamination, such as at the entry points to the home, for example at front and back doors. In use, the disinfection process is realized by the user from standing on the apparatus for a few seconds. During this time the outer soles and heels of the footwear or other items or the feet are exposed to germicidal ultraviolet light. The timed exposure effectively kills the microorganisms present on the surface of the exposed footwear or other items or a user's feet, soles and heels.

The apparatus may also include means of manually activating the ultraviolet lamps instead or as an augment to the weight activated means for activating the lamps. This manual activation may be employed in order to sanitize the bottom of footwear while they are not being worn, or to also disinfect other outer surfaces of the footwear besides the outer soles and heels, or to sanitize the external surfaces of other items that may require disinfection such as ladies purses, backpacks, and other items that may have been placed on unsanitary surfaces. Of course any such manual switch should be provided in a manner that the user is not exposed to the UV light during activation of the switch.

In another preferred embodiment, the device is as described in the preferred embodiment above, however with an additional housing on top is provided. The housing is adapted for insertion of a foot or other article insertion therein using an opening formed on one vertical side which continues into a slot formed in the top surface of the housing which is spaced from the platform. The end opening and slot allow a user to step onto the platform and slide their feet or foot under the housing when using the device. Employment of this mode of the device provides an additional means to preventing stray radiation as an additional safety measure. This same mode of the device may also be constructed using one or more additional ultraviolet lamps under the top wall of the housing and above the platform. The additional lamps will provide the additional advantage that all outer surfaces can be exposed to the radiation simultaneously thereby allowing all outer surfaces of the footwear or other items or feet to be disinfected concurrently in one timed exposure. Alternatively, the additional ultraviolet lamps under the top housing may be supplemented or may replaced by mirrors to direct the radiation from sources under the platform downward onto the exposed upper surfaces of the user's feet or shoes.

In another preferred embodiment, the device is as described above, however with the additional provision of mirrors in angled positioning within the bottom housing of the device below the platform, to more effectively direct the radiation to the sides of the footwear or other items or feet that are irradiated. The angled lower mirrors may also direct radiation to mirrors above the platform and under the upper housing to irradiate the sides and tops of feet or footwear of the user.

In all preferred modes of the device, the user may be provided with a signaling component to indicate the device is in an energized state, and therefor emitting UV or other light. Since UV light is invisible to the human eye, but could harm it, the signaling component to provide the user with a warning is thus preferred. Preferred means for signaling may be a means for signaling which includes one or a combination of individual signaling components, from a group consisting of an audible alarm, a light emitter, and a vibrator which is in communication with said platform. Any one or combination of these signaling components thereby provides the user with a means to ascertain that UV light is emanating from the device.

It is thus an object of this invention to provide a device for disinfecting the outer external bottom surfaces of footwear and other items which is simple, quick, compact and convenient to use.

It is a further object of this invention to provide such a device for disinfecting the outer external bottom surfaces of footwear and other items and disinfecting feet, which does not require the use of liquids or spray-on cleaning chemicals.

It is another object of this invention to provide a device for disinfecting the outer external bottom surfaces of footwear and other items and disinfecting feet which does not require employment of potentially toxic pesticides, such as disinfectants.

It is yet a further object of this invention to provide such a device for disinfecting the outer external bottom surfaces of footwear and other items and disinfecting feet which does not require the use of brushes, physical scrubbing or other physical cleaning actions thereby encouraging use.

It is an additional object of this invention to provide a device for disinfecting the outer external bottom surfaces of footwear and other items and disinfecting feet which employs radiation sterilization which has safety features to ensure it can be used safely.

A still further object of this invention is to provide a device for disinfecting the outer external bottom surfaces of footwear and other items and disinfecting feet which is both timed and weight activated when a person steps on a platform, thereby initiating the disinfection process when a platform is stepped upon, and ceasing such when the platform is vacated or a predetermined time has elapsed.

Yet another object of this invention is to provide a device for disinfecting the outer external bottom surfaces of footwear and other items and disinfecting feet which employs as a safety component, a weight activated means for switching which requires minimum weight to activate the device's radiation source to ensure children and pets who weigh less than that minimum, cannot activate the device without adult supervision.

An additional object of this invention is to provide a device for disinfecting the outer external bottom surfaces of footwear and other items and disinfecting feet which includes a manual means for activation to allow use for other articles or to override the minimum weight requirements of the weight activated means for activation.

Another object of this invention is to provide a device for disinfecting the outer external bottom surfaces of footwear and other items and disinfecting feet which has a manual activation and a timer to insure that the radiation source will be switched off after the required exposure time has elapsed.

A still further object of this invention is to provide a device for disinfecting the outer external bottom surfaces of footwear and other items and disinfecting feet which is adapted to accommodate objects having a wide variety of shapes and sizes.

An additional object of this invention is to provide a device for disinfecting other external surfaces of footwear and other items besides the bottom surfaces, which will accommodate longer exposure times by providing a manual means for activation which may override a timed means for cessation of the radiation.

A further object of this invention is to provide a device for disinfecting the outer external bottom surfaces which is adapted in a configuration to accommodate such items of various odd shapes and sizes and which may include hand bags, purses, backpacks, duffel bags, as well as other items.

Another object of this invention is to provide a device for disinfecting the outer external surfaces of footwear other items and disinfecting feet which maximizes deployment and use safely by minimizing any required maintenance of the device.

These and further objectives of this invention will be brought out in the following part of the specification, wherein detailed description is for the purpose of fully disclosing the invention without placing limitations thereon.

With respect to the description provided herein, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. Therefore, the descriptions provided herein are considered as illustrative only of the principles of the invention. Further, since numerous modifications and changes will readily occur to those skilled in the art upon reading this disclosure, it is not desired to limit the invention to the exact construction and operation shown and described, and accordingly, all suitable modifications and equivalents which may be resorted to, are considered to be within the scope of this invention.

### BRIEF DESCRIPTION OF THE DRAWINGS

The accompanying drawings, which are incorporated in and form a part of this specification, illustrate preferred embodiments of the invention and together with the description, serve to explain the principles of this invention.

FIG. 1 shows a perspective view of the apparatus;

FIG. 2 is a sectional view of the apparatus taken along the Line 2-2 of FIG. 1;

FIG. 3 depicts a plan view of the apparatus;

FIG. 4 is a perspective view of the apparatus showing one of the ways in which it can be used.

FIGS. 5, 6 and 7 are perspective views of the apparatus showing examples of how it can be used;

FIG. 8 is a perspective view of another mode of the disclosed device herein;

FIG. 9. depicts a sectional view of the device as shown in FIG. 8 taken along the line 15-15;

FIG. 10 depicts an elevation view of the device as shown in FIG. 8.

### DETAILED DESCRIPTION OF THE PREFERRED EMBODIMENTS OF THE INVENTION

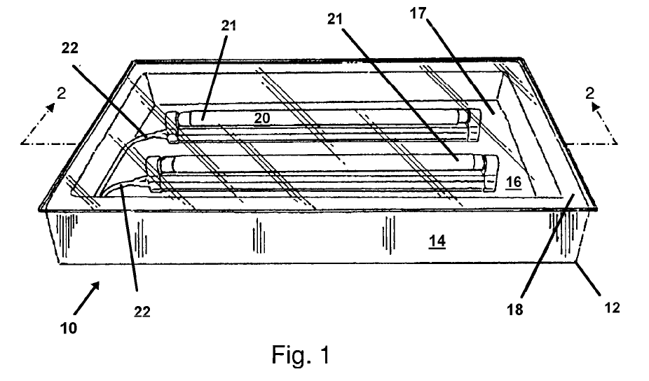

Referring now to the drawings and FIGS. 1-10, wherein similar parts of the invention are identified by like reference numerals in one or more of the different drawings, there are illustrated various preferred embodiments of the device 10. It is to be understood that other embodiments may be utilized and structural changes may be made without departing from the scope of the present invention and any and all changes or adaptations which may be made by those skilled in the art, are considered to be within the scope of the claimed device and method.

There is seen in FIG. 1 a perspective view of the disclosed device 10. The device 10 employs housing 12 which is formed by vertical sidewalls 14 extending between a bottom wall 16 and a platform 18 held elevated above the bottom wall 16 and defining an internal cavity 17 under the platform 18.

The platform 18 of the device 10 is preferably formed of a substantially transparent planar material which is adapted to provide a means to control transmission of radiated light from a light source in the cavity 17 through the platform 18. Particularly preferred in this embodiment is a light source which radiates ultraviolet UV-C radiation in the 100-280 nm range. The platform 18 while formed of substantially transparent material which may be structured to control light transmission therethrough, needs to also be structurally strong such that it will support the weight of a person standing on the platform 18 during use.

As depicted, a means for germicidal radiation 20 is positioned in the cavity 17 and as depicted would employ one or a plurality of UV-C germicidal lamps 21, which are connected by electrical wires 22 to wires leading through a switch 23 to wires from a power source 25 such as AC power in a home or office or a battery pack but which is not shown in the drawings.

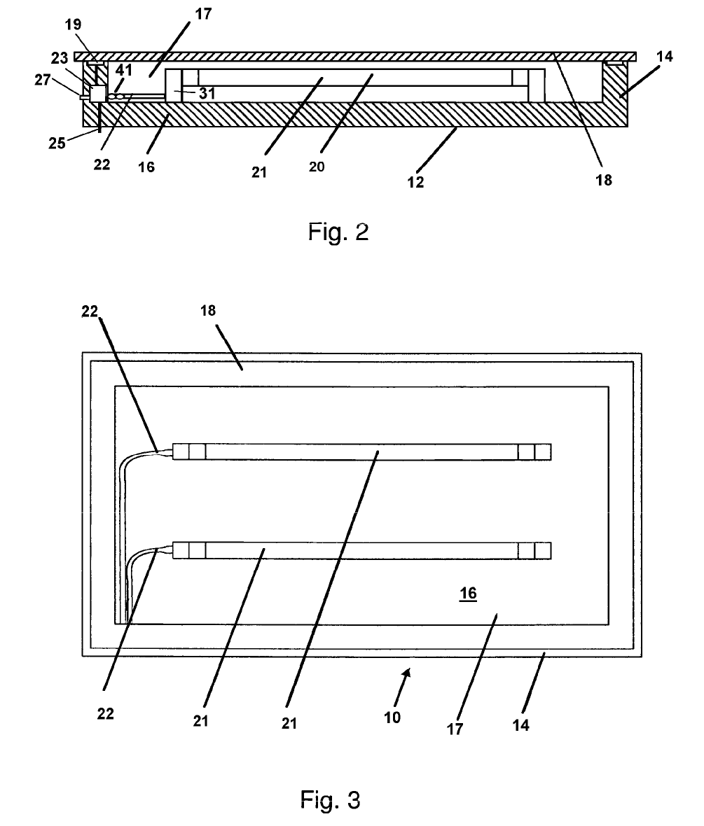

FIG. 2 shows a sectional view taken along the line 2-2 of FIG. 1 and depicts the vertical sidewalls 14 of the housing 12, the bottom wall 16 of the housing 12, and the platform 18. Also shown is one of the ultraviolet germicidal lamps 21 operatively communicating through wires 22 engaged through a switch 23 with wires leading to a power source 25 which is adapted to energize the lamps 21. Lamps 21 or other radiation sources are energized to an illuminated state when force of the weight of the person stepping onto the platform 18 is sufficient to close a weight sensing component 19 which will activate a switch 23 to communicate power from the wires leading to a power source 25, to the lamps 21. The weight sensing component 19 might be electronic in nature or may be a mechanical switch or may be other means to close the circuit to communicate power to the lamps 21 when a predetermined weight is sensed on the platform 18 or any other such means for weight switching as would occur to those skilled in the art.

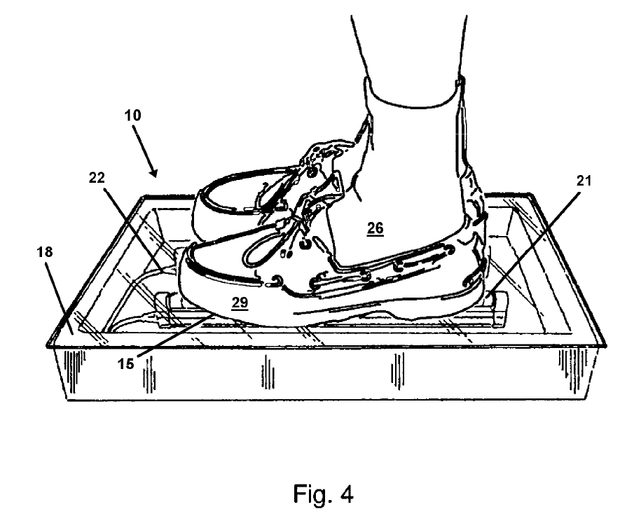

FIG. 3 depicts a plan view of the device 10, showing the housing 12, consisting of the vertical sidewalls 14 and the bottom wall 16, and the platform 18 supported over the sidewalls 14 and above the cavity 17. In the cavity 17 are housed the ultraviolet lamps 21 or other means for generating germicidal radiation which are operatively engaged with wiring 22 communicating through a switch 23 with wires leading to a power source 25.

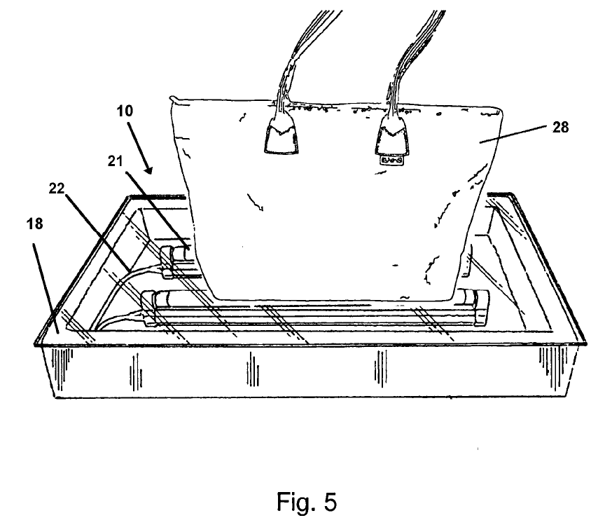

FIG. 4 shows a perspective view of the device 10 similar to that of FIG. 1 in the as-used position with a person standing on the platform 18. FIG. 4 illustrates a principal mode of use of the device 10, namely, illuminating the footwear while supported on the platform 18. Such use provides a quick and effective means of sanitizing the external soles and heels of footwear 29 without the need to remove the footwear 29 from the feet or use scrubbing or liquids. In use, the person wishing to disinfect the exterior bottom surfaces of his or her footwear 29, steps onto the platform 18 preferably after first wiping his or her footwear 29 on a doormat to remove any dirt, soil or other foreign materials adhering to the bottom of the footwear 29.

Also shown in FIG. 4 depicts the legs and feet 26 of the person wearing shoes standing on the device 10. Energizing the means for germicidal radiation such as germicidal lamps 21, by turning on the electrical power communicated to the electrical wiring 22, results in a transmission of germicidal ultraviolet radiation through the platform 18 to contact the outer bottom surfaces of the footwear 29 while still on the feet 26 of the user. In this very basic mode of the device 10 activation to communicate electrical power to the lamps 21 or other germicidal radiation source, may be accomplished manually by a button 27 closing the switch 23 or other means to manually close the switch 23 interrupting electrical power to the lamps 21 to cause power to communicate and illuminate the lamps 21. However if that be the case, the button 27 should be positioned to make sure the user's eyes are shielded from the UV radiation through the platform by a cover or the like, or by provision and use of safety glasses (not shown) which employ lenses which block the light from the lamps 21 from reaching the eye.

In a particularly preferred mode of the device 10, the activation of the germicidal lamps 21 or other radiation source, is achieved by the force of the weight of the person stepping onto the platform 18. Means for weight activation of the device 10 to an illuminated state, with the lamps 21 energized, is provided by a weight sensitive means for switching 19. Such a weight sensitive means for switching 19 may be provided by one or more load cells, a spring loaded switch, or other means of converting force of the mass of a user on the platform 18, into an electrical signal or communication which when connected in an appropriate circuit between a power source 25 and the lamps 21, can serve as the input for electronically switching the germicidal lamps 21 to the energized state.

Most preferably the weight sensitive means for switching 19 is adjustable in that the employed switch will not activate the lamps 21, to an energized state when unsupervised children step on the device 10, as their weight would be below a threshold weight set for activation. This preferred embodiment also incorporates an electronic timer mechanism 31 to turn off the germicidal lamps 21 after the required exposure time has elapsed.

Further, a preferred embodiment of the device also provides the means of a switch button 27 of the germicidal lamps 21 along with the timer mechanism 31 to switch off the lamps after the required, preset exposure time has elapsed. This feature allows for the use of the device 10 to sanitize footwear while not being worn as well as other items such as handbags, backpacks, etc.

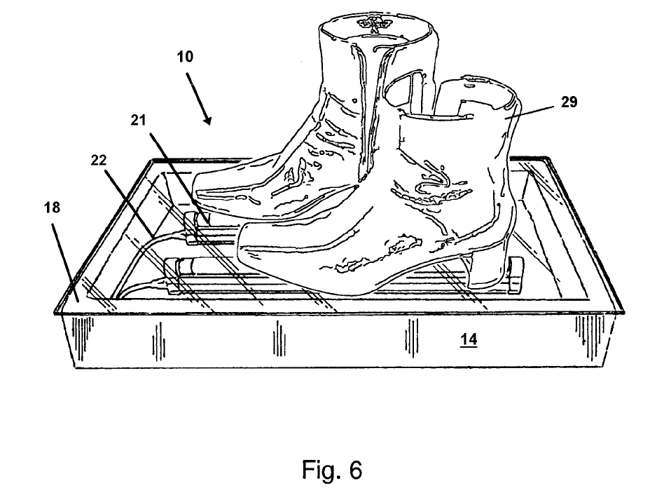

FIGS. 5 and 6 show perspective views of the device 10 similar to the view as shown in FIG. 1, illustrating an alternate manner to employ the device 10 to sanitize the bottom of footwear 15 while they are not being worn, or to also disinfect other outer surfaces of the footwear 29 besides the outer soles and heels, or to sanitize the external surfaces of other items that may require disinfection such as ladies purses 28 and other items that may have been placed on unsanitary surfaces.

FIG. 7 shows a perspective view of the device 10 similar to the view as shown in FIG. 1, as well as a person standing barefoot on the platform and illustrating another application of the apparatus, namely a quick, easy and effective means of sanitizing a person's feet 26 to treat foot infections such as athlete's foot.

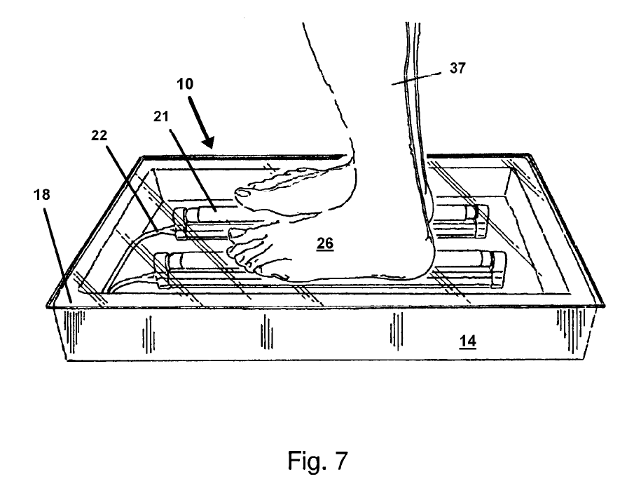

FIG. 8 shows a perspective view of the device 10, which has been fitted with another component in the form of a second housing 30 on top which has a slot 33 communicating through one vertical side 14 and the top horizontal wall 35 of the second housing 30 thereby allowing the user to step into the second housing 30 when using the device 10. This embodiment has the advantage of preventing stray radiation as an additional safety measure.

This same embodiment of the device 10 can also be constructed with the use of one or more additional ultraviolet lamps 21 within the top housing 30, which provides as additional advantage that all outer surfaces can be exposed to the radiation simultaneously and allowing all outer surfaces of the footwear or other items or feet to be disinfected. Alternatively, the additional ultraviolet lamps 21 in the top housing may be supplemented or replaced by mirrors 32 to direct the radiation downward.

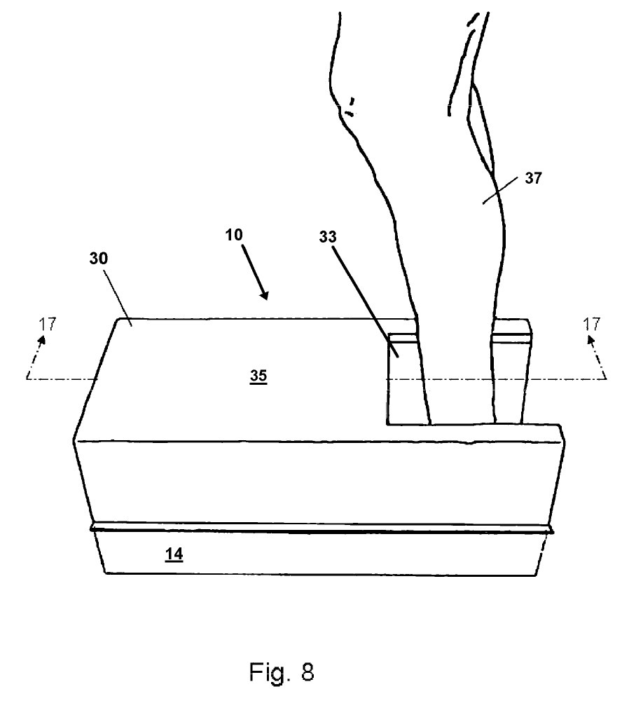

FIG. 9 shows a sectional view taken along the line 17-17 of FIG. 8 revealing the device 10 similar as to that illustrated in FIG. 2, and with the second housing 30 and mirrors 32.

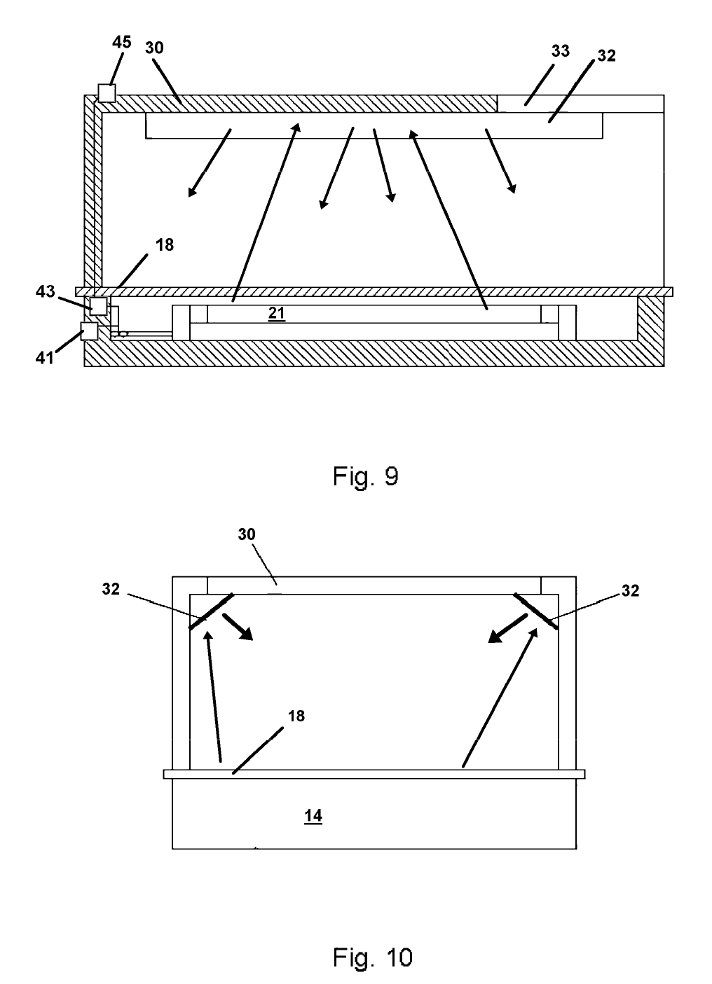

FIG. 10 shows an elevation view of the embodiment illustrated in FIG. 8, revealing the device 10, the additional housing 30 and the mirrors 32.

As noted above, in all preferred modes of the device 10, the user is preferably provided with a means to determine that the device is energized and emitting sanitizing UV light since such is invisible to the human eye and potentially harmful. A preferred means for signaling the user of the device being energized, may be a means for signaling which includes one or a combination of individual signaling components, from a group consisting of an audible alarm 41, a light emitter 45, and a vibrator 43 which is in communication with said platform. All such means for signaling operation would be operatively wired to operate during illumination of the germicidal lamps 21.

The device to sanitize the external bottom surfaces of footwear as shown in the drawings and described in detail herein discloses arrangements of elements of particular construction and configuration for illustrating preferred embodiments of structure and method of operation of the present invention. It is to be understood, however, that elements of different construction and configuration and other arrangements thereof, other than those illustrated and described may be employed for providing an apparatus to sanitize the external bottom surfaces of footwear in accordance with the spirit of this invention, and such changes, alternations and modifications as would occur to those skilled in the art are considered to be within the scope of this invention as broadly defined in the appended claims.

What is claimed is:

1. An apparatus for disinfecting objects placed thereon, comprising:
a platform, said platform having an upper surface and a lower surface and formed of substantially transparent material therebetween, said platform adapted to support and object upon said upper surface;
means to support said platform above a support surface and adapted to support an object thereon;
a cavity defined by the area between said support surface and said platform; and
means for illumination located in said cavity, said means for illumination radiating light in a spectral range adapted to communicate through said transparent material and to sanitize said object supported upon said platform, whereby a said object such as a foot or shoe, supported on said platform, is sanitized on surfaces facing said upper surface by said light communicated from said cavity.

2. The disinfecting apparatus of claim 1 additionally comprising:
four vertically inclined sidewalls surrounding said cavity; and said sidewalls formed on opaque material preventing said light from transmission therethrough thereby providing means to prevent said light from emanating from said cavity through said sidewalls.

3. The disinfecting apparatus in claim 1, wherein said means for illumination emits at least a portion of light in an ultraviolet said spectral range.

4. The disinfecting apparatus in claim 2, wherein said means for illumination emits at least a portion of light in an ultraviolet said spectral range.

5. The disinfecting apparatus in claim 3, wherein the radiation source emits ultraviolet light in said spectral range of UV-C between 100 to 280 nm.

6. The disinfecting apparatus in claim 4, wherein the radiation source emits ultraviolet light in said spectral range of UV-C between 100 to 280 nm.

7. The disinfecting apparatus in claim 5, wherein said platform above said means for illumination radiation is made of glass.

8. The disinfecting apparatus in claim 6, wherein the platform above said means for illumination radiation is made of glass.

9. The disinfecting apparatus in claim 5 wherein the glass is of a composition which blocks a certain portion of the UV radiation from communicating through said platform, thereby providing a filter permitting only a remainder portion of said UV radiation from illuminating said object.

10. The disinfecting apparatus in claim 6 wherein the glass is of a composition which blocks a certain portion of the UV radiation from communicating through said platform, thereby providing a filter permitting only a remainder portion of said UV radiation from illuminating said object.

11. The disinfecting apparatus claim 5, wherein the platform above the radiation source is made of an open metal grid having apertures therein, said apertures providing said transparent material.

12. The disinfecting apparatus of claim 6, wherein the platform above the radiation source is made of an open metal grid having apertures therein, said apertures providing said transparent material.

13. The disinfecting apparatus of claim 7 additionally comprising:
a switch, said switch being activated to energize said means for illumination by weight on said platform only when said weight exceeds a threshold;
said threshold being a said weight above that of a child in residence with said disinfecting apparatus; and
a timer, said timer de-energizing said means for illumination subsequent to the elapse of a time duration, and whereby said switch provides means to prevent use of said apparatus by a child and said timer provides means to prevent exposure of said object to said light for a time period exceeding said time duration.

14. The disinfecting apparatus of claim 8 additionally comprising:
a switch, said switch being activated to energize said means for illumination by weight on said platform only when said weight exceeds a preset threshold;
said threshold being a said weight above that of a child, said threshold determinable by a user; and
a timer, said timer de-energizing said means for illumination subsequent to the elapse of a time duration, and whereby said switch provides means to prevent use of said apparatus by a child and said timer provides means to prevent exposure of said object to said light for a time period exceeding said time duration.
said weight only when said object on said platform exceeds a the above claims, wherein the means for illumination is activated to emit said light by the weight of the person standing on the platform and the weight has to be above a certain minimum level to preclude unsupervised young children from using the apparatus, and said means for illumination is deactivated through means of a timer which turns the radiation source off after the required radiation exposure time has elapsed.

15. The disinfecting apparatus of claim 7 additionally comprising:
a cover, said cover formed of opaque material preventing transmission of said light therethrough;
said cover having a sidewall surrounding said platform and an elevated topwall connected to said sidewall;
an upper cavity formed between said top surface, said sidewall, and said topwall;
an aperture formed in said sidewall providing a communication with said upper cavity;
said sidewall and said topwall providing means to prevent light transmission from light communicated through said platform; and
whereby a user placing a foot through said aperture and supporting it on said upper surface in an as-used position, is provided a protection by said top wall from a direct communication to their eyes from said light communicating through said platform.

16. The disinfecting apparatus of claim 8 additionally comprising:
a cover, said cover formed of opaque material preventing transmission of said light therethrough;
said cover having a sidewall surrounding said platform and an elevated topwall connected to said sidewall;
an upper cavity formed between said top surface, said sidewall, and said topwall;
an aperture formed in said sidewall providing a communication with said upper cavity;
said sidewall and said topwall providing means to prevent light transmission from light communicated through said platform; and
whereby a user placing a foot through said aperture and supporting it on said upper surface in an as-used position, is provided a protection by said top wall from a direct communication to their eyes from said light communicating through said platform.

17. The disinfecting apparatus of claim 13 additionally comprising:
a cover, said cover formed of opaque material preventing transmission of said light therethrough;
said cover having a sidewall surrounding said platform and an elevated topwall connected to said sidewall;
an upper cavity formed between said top surface, said sidewall, and said topwall;
an aperture formed in said sidewall providing a communication with said upper cavity;
said sidewall and said topwall providing means to prevent light transmission from light communicated through said platform; and
whereby a user placing a foot through said aperture and supporting it on said upper surface in an as-used position, is provided a protection by said top wall from a direct communication to their eyes from said light communicating through said platform.

18. The disinfecting apparatus of claim 14 additionally comprising:
a cover, said cover formed of opaque material preventing transmission of said light therethrough;
said cover having a sidewall surrounding said platform and an elevated topwall connected to said sidewall;
an upper cavity formed between said top surface, said sidewall, and said topwall;
an aperture formed in said sidewall providing a communication with said upper cavity;
said sidewall and said topwall providing means to prevent light transmission from light communicated through said platform; and
whereby a user placing a foot through said aperture and supporting it on said upper surface in an as-used position, is provided a protection by said top wall from a direct communication to their eyes from said light communicating through said platform.

19. The disinfecting apparatus of claim 17 additionally comprising:
a signaling component, said signaling component including one or a combination of signaling components from a group consisting of an audible alarm, a light emitter, and a vibrator in communication with said platform, whereby a user is provided with a means to discern said UV light is communicating through said platform without directly looking at said platform.

20. The disinfecting apparatus of claim 18 additionally comprising:
a signaling component, said signaling component including one or a combination of signaling components from a group consisting of an audible alarm, a light emitter, and a vibrator in communication with said platform, whereby a user is provided with a means to discern said UV light is communicating through said platform without directly looking at said platform.

21. The disinfecting apparatus of claim 17 additionally comprising:
said cover having an interior surface facing said platform, at least a portion of said interior surface formed of reflective material; and
said reflective material directing said light to upper surfaces of said object.

22. The disinfecting apparatus of claim 18 additionally comprising:
said cover having an interior surface facing said platform, at least a portion of said interior surface formed of reflective material; and
said reflective material directing said light to upper surfaces of said object.
